(19) United States
(12) Patent Application Publication
(10) Pub. No.: US 2014/0081301 A1
(43) Pub. Date: Mar. 20, 2014

(54) NERVE MODULATION SYSTEM

(71) Applicant: BOSTON SCIENTIFIC SCIMED, INC., Maple Grove, MN (US)

(72) Inventors: BINH C. TRAN, MINNEAPOLIS, MN (US); ROGER N. HASTINGS, MAPLE GROVE, MN (US); MARK L. JENSON, GREENFIELD, MN (US); LEONARD B. RICHARDSON, BROOKLYN PARK, MN (US); MICHAEL J. PIKUS, GOLDEN VALLEY, MN (US)

(73) Assignee: BOSTON SCIENTIFIC SCIMED, INC., Maple Grove, MN (US)

(21) Appl. No.: 14/029,452

(22) Filed: Sep. 17, 2013

Related U.S. Application Data
(60) Provisional application No. 61/702,048, filed on Sep. 17, 2012.

(51) Int. Cl. A61B 17/32

(52) U.S. Cl. CPC A61B 17/320068
USPC 606/169

(57) ABSTRACT

Systems for nerve and tissue modulation are disclosed. An example system may include a first elongate element having a distal end and a proximal end and having at least one nerve modulation element disposed adjacent the distal end. The nerve modulation element may be positioned or moveable to target a particular tissue region. The nerve modulation element may be an ultrasound transducer.

### CROSS-REFERENCE TO RELATED APPLICATIONS

This application claims priority under 35 U.S.C. §119 to U.S. Provisional Application Ser. No. 61/702,048, filed Sep. 17, 2012, the entirety of which is incorporated herein by reference.

### TECHNICAL FIELD

The present invention relates to methods and apparatuses for nerve modulation techniques such as ablation of nerve tissue or other destructive modulation technique through the walls of blood vessels and monitoring thereof.

### BACKGROUND

Certain treatments require the temporary or permanent interruption or modification of select nerve function. One example treatment is renal nerve ablation which is sometimes used to treat conditions related to congestive heart failure or hypertension. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves, including renal nerves, run along the walls of or in close proximity to blood vessels and thus can be accessed via the blood vessels. In some instances, it may be desirable to ablate perivascular renal nerves using ultrasound energy in an off-wall configuration. In an off-wall configuration, tissue changes may be monitored with imaging transducers. However, in some instances, ablated tissue may attenuate ultrasound energy. When a portion of tissue is ablated, tissue properties change and increased attenuation of the ultrasound energy can make ablation past this ablated tissue difficult. Attenuation of the ultrasound energy may require extended treatment for complete ablation which may risk injury to the artery wall. It may be desirable to provide for alternative systems and methods for intravascular nerve modulation for reducing problems associated with tissue attenuation.

### SUMMARY

The disclosure is directed to several alternative designs, materials and methods of manufacturing medical device structures and assemblies for performing nerve ablation.

Accordingly, one illustrative embodiment is a system for nerve modulation that may include an elongate shaft having a proximal end region and a distal end region. An array of ultrasound ablation transducers may be positioned at the distal end region. In some instances, the system may further include one or more imaging transducers. The ablation transducers may be arranged to target different focal points of a target region. Alternatively, or additionally, the system may include mechanisms to change the focal points of the ablation transducers. Such mechanisms may include, but are not limited to tension ribbons, shape memory materials, and/or inflatable members. Acoustic energy may be radiated from the ablation transducers to perform tissue ablation as desired.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a distal end of an illustrative renal nerve modulation system.

FIG. 3 illustrates a distal end of another illustrative renal nerve modulation system.

FIG. 4 illustrates a distal end of another illustrative renal nerve modulation system.

FIG. 5 is another illustrative view of the renal nerve modulation system of FIG. 4.

FIG. 6A illustrates a distal end of another illustrative renal nerve modulation system.

FIG. 6B is another illustrative view of the renal nerve modulation system of FIG. 6A.

FIG. 7A illustrates a distal end of another illustrative renal nerve modulation system.

FIG. 7B is another illustrative view of the renal nerve modulation system of FIG. 7A.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, tumor ablation, benign prostatic hyperplasia therapy, nerve excitation or blocking or ablation, modulation of muscle activity, hyperthermia or other warming of tissues, etc. In some instances, it may be desirable to ablate perivascular renal nerves with ultrasound ablation.

Ultrasound energy may be a safer, more consistent, and more efficient method of performing tissue ablation than radiofrequency (RF) ablation. The target nerves must be heated sufficiently to make them nonfunctional, while thermal injury to the artery wall is undesirable. Heating of the artery wall may also increase pain during the procedure. When a portion of tissue is ablated, tissue properties change and increased attenuation of the ultrasound energy can make ablation past this ablated tissue difficult. An array of multiple ultrasound transducers physically directed towards a single focal region may be an efficient method to target deeper tissue first, followed by shallower tissue, avoiding problems with tissue attenuation. This efficiency enables use of fewer transducers and/or lower power.

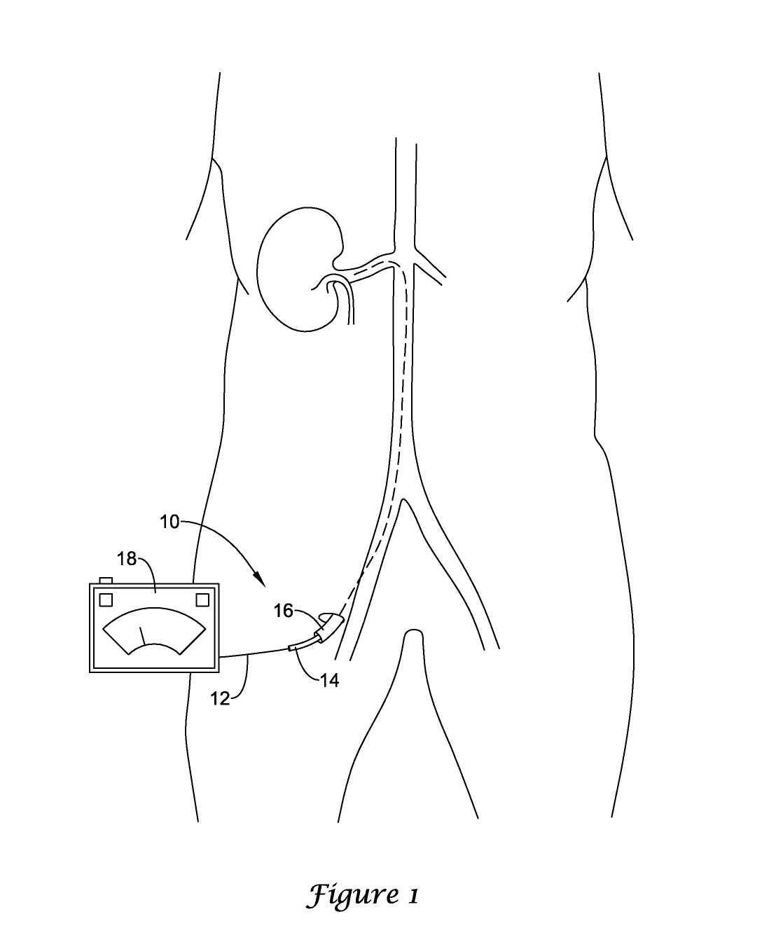

FIG. 1 is a schematic view of an illustrative renal nerve modulation system 10 in situ. System 10 may include an element 12 for providing power to a transducer disposed adjacent to, upon, about, and/or within a central elongate shaft 14 and, optionally, within a sheath 16, the details of which can be better seen in subsequent figures. A proximal end of element 12 may be connected to a control and power element 18, which supplies the necessary electrical energy to activate the one or more transducers at or near a distal end of the element 12. The control and power element 18 may include monitoring elements to monitor parameters such as power, temperature, voltage, and/or frequency and other suitable parameters as well as suitable controls for performing the desired procedure. In some instances, the power element 18 may control an ultrasound transducer. The transducer may be configured to operate at a frequency of approximately 9-10 megahertz (MHz). It is contemplated that any desired frequency may be used, for example, from 1-20 MHz. However, it is contemplated that frequencies outside this range may also be used, as desired. While the term “ultrasound” is used herein, this is not meant to limit the range of vibration frequencies contemplated.

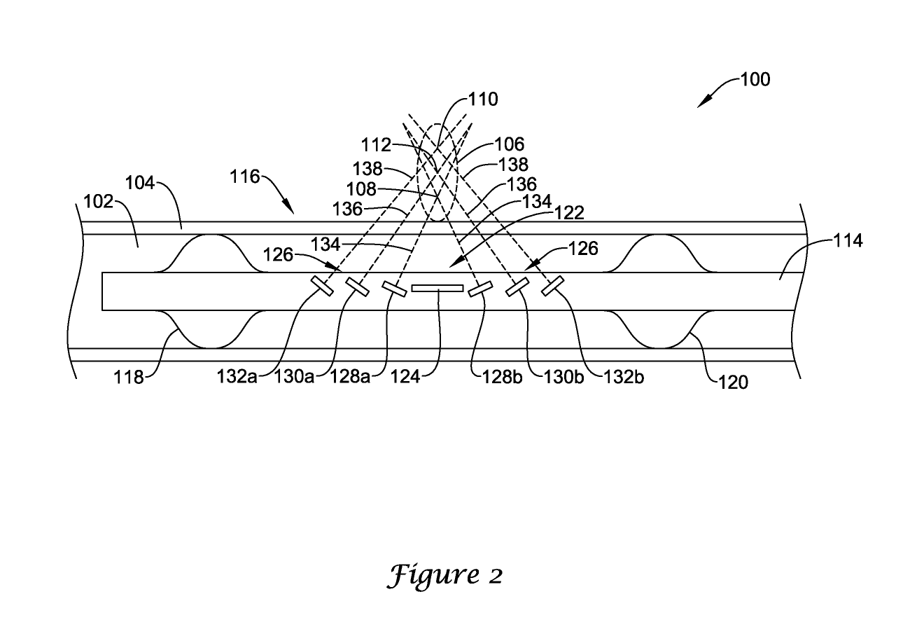

FIG. 2 is an illustrative embodiment of a distal end of a renal nerve modulation system 100 disposed within a body lumen 102 having a vessel wall 104. The vessel wall 104 may be surrounded by local body tissue. The local body tissue may comprise adventitia and connective tissues, nerves, fat, fluid, etc. in addition to the muscular vessel wall 104. A portion of the tissue may be the desired treatment region 106 having a shallow region 108 adjacent to the vessel wall 104, a deeper region 110, and a middle region 112 disposed between the shallow region 108 and the deeper region 110. As will become more apparent below, it is contemplated that there may be any number of sub-regions within the target region 106. The number of sub-regions may be determined by the number and relative position of ablation transducers disposed on the elongate shaft 114. The system 100 may include an elongate shaft 114 having a distal end region 116. The modulation system 100 may include one or more expandable centering baskets or framework 118, 120 disposed adjacent the distal end region 116. In some instances the modulation system 100 may include expandable balloon or other centering device in place of the expandable basket(s) 118, 120. It is contemplated that a first expandable basket 118 may be positioned distal to the transducer array 122 and a second centering basket 120 may be placed proximal to the transducer array 122.

The elongate shaft 114 may extend proximally from the distal end region 116 to a proximal end configured to remain outside of a patient's body. The proximal end of the elongate shaft 114 may include a hub attached thereto for connecting other treatment devices or providing a port for facilitating other treatments. It is contemplated that the stiffness of the elongate shaft 114 may be modified to form a modulation system 100 for use in various vessel diameters and various locations within the vascular tree. The elongate shaft 114 may further include one or more lumens extending therethrough. For example, the elongate shaft 114 may include a guidewire lumen and/or one or more auxiliary lumens. The lumens may be configured in any way known in the art. For example, the guidewire lumen may extend the entire length of the elongate shaft 114 such as in an over-the-wire catheter or may extend only along a distal portion of the elongate shaft 114 such as in a single operator exchange (SOE) catheter. These examples are not intended to be limiting, but rather examples of some possible configurations. While not explicitly shown, the modulation system 100 may further include temperature sensors/wire, an infusion lumen, radiopaque marker bands, fixed guidewire tip, a guidewire lumen, external sheath and/or other components to facilitate the use and advancement of the system 100 within the vasculature.

The system 100 may include an array of transducers 122. In some embodiments, the array may include one or more optional imaging transducers 124 and one or more ultrasound ablation transducers 126 disposed adjacent the distal end region 116. However, the transducer array 122 may be placed at any longitudinal location along the elongate shaft 114 desired. In some embodiments, should one be so provided, the one or more imaging transducers 124 may be provided at the center of the array 122 to detect tissue changes during the ablation procedure. However, the imaging transducer 124 may be provided at any location within the array desired. In some instances, the ablation transducers 126 may be placed symmetrically about the imaging transducer 124 such that there is equal number of transducers 126 located proximal to the imaging transducer 124 and distal to the imaging transducer 124. However the ablation transducers 126 may be arranged in any pattern desired. For example, in some instances, there may not be an equal number of ablation transducers 126 disposed on either side of the imaging transducer 124. It is further contemplated that in some embodiments, the imaging transducer 124 may not be present. It is contemplated that the transducer array 122 may include any number of imaging transducers 124 and ablation transducers 126 desired. It is further contemplated that more than one row of transducers 122 may be disposed on the elongate shaft 114.

The ablation transducers 126 may be formed from any suitable material such as, but not limited to, lead zirconate titanate (PZT). It is contemplated that other ceramic or piezoelectric materials may also be used. While not explicitly shown, the ablation transducers 126 may have a first radiating surface, a second radiating surface, and a perimeter surface extending around the outer edge of the ablation transducer 126. In some instances, the transducers 126 may include a layer of gold, or other conductive layer, disposed on the first and/or second side over the PZT crystal for connecting electrical leads to the transducers 126. In some embodiments, the ablation transducers 126 may be structured to radiate acoustic energy from a single radiating surface. In such an instance, one radiating surface may include a backing layer to direct the acoustic energy in a single direction. In other embodiments, the ablation transducers 126 may be structured to radiate acoustic energy from two radiating surfaces. In some instances, one or more tie layers may be used to bond the gold to the PZT. For example, a layer of chrome may be disposed between the PZT and the gold to improve adhesion. In other instances, the transducers 126 may include a layer of chrome over the PZT followed by a layer of nickel, and finally a layer of gold. These are just examples. It is contemplated that the layers may be deposited on the PZT using sputter coating, although other deposition techniques may be used as desired.

It is contemplated that the radiating surface (surface which radiates acoustic energy) of the transducers 126 may take any shape desired, such as, but not limited to, square, rectangular, polygonal, circular, oblong, etc. The acoustic energy from the radiating surface of the transducers 126 may be transmitted in a spatial pressure distribution related to the shape of the transducers 126. With exposures of appropriate power and duration, lesions formed during ablation may take a shape similar to the contours of the pressure distribution. As used herein, a “lesion” may be a change in tissue structure or function due to injury (e.g. tissue damage caused by the ultrasound). Thus, the shapes, dimensions, and arrangement of the transducers 126 may be selected based on the desired treatment and the shape best suited for that treatment. It is contemplated that the transducers 126 may also be sized according to the desired treatment region. For example, in renal applications, the transducers 126 may be sized to be compatible with a 6 French guide catheter, although this is not required.

In some embodiments, the transducers 126 may be formed of a separate structure and attached to the elongate shaft 114. For example, the transducers 126 may be bonded or otherwise attached to the elongate shaft 114. In some instances, the transducers 126 may include a ring or other retaining or holding mechanism (not explicitly shown) disposed around the perimeter of the transducers 126 to facilitate attachment of the transducers 126. The transducers 126 may further include a post, or other like mechanism, affixed to the ring such that the post may be attached to the elongate shaft 114 or other member. In some instances, the rings may be attached to the transducers 126 with a flexible adhesive, such as, but not limited to, silicone. However, it is contemplated that the rings may be attached to the transducers 126 in any manner desired. While not explicitly shown, in some instances, the elongate shaft 114 may be formed with grooves or recesses in an outer surface thereof. The recesses may be sized and shaped to receive the transducers 122. For example, the ablation transducers 126 may be disposed within the recess such that a first radiating surface contacts the outer surface of the elongate shaft 114 and a second radiating surface is directed towards a desired treatment region. However, it is contemplated that the transducers 122 may be affixed to the elongate shaft in any manner desired.

In some embodiments, the transducers 122 may be affixed to an outer surface of the elongate shaft 114 such that the surfaces of the transducers 122 are exposed to blood flow through the vessel. As the power is relayed to the ablation transducers 126, the power that does not go into generating acoustic power generates heat. As the ablation transducers 126 heat, they become less efficient, thus generating more heat. Passive cooling provided by the flow of blood may help improve the efficiency of the transducers 126. As such, additional cooling mechanisms may not be necessary. However, in some instances, additional cooling may be provided by introducing a cooling fluid to the modulation system.

The transducer array 122 may include multiple ultrasound ablation transducers 126 physically directed towards multiple focal points. In some embodiments, more than one ablation transducer 126 may be directed towards a single focal point. In some instances, this arrangement may be more efficient than focusing by phased arrays of linearly arranged transducer elements that are not physically directed at the focal point. It is contemplated that an increased efficiency resulting from multiple ablation transducers physically directed towards single focal point may enable the use of fewer transducers and/or lower power. The modulation system 100 may include a first pair of ablation transducers 128a, 128b (collectively 128a,b) directed towards the shallow region 108 of the desired treatment region 106, a second pair of transducers 130a, 130b (collectively 130a,b) directed towards the middle region 112 of the desired treatment region 106, and a third pair of transducers 132a, 132b (collectively 132a,b) directed towards the deeper region 110 of the desired treatment region 106. While the system 100 is described as having three pairs of ablation transducers 128a,b, 130a,b, 132a,b it is contemplated that the system 100 may include any number of transducers (or pairs of transducers), such as, but not limited to: one, two, three, four, or more depending on the number of desired target regions. It is further contemplated that the system 100 may have any number of ablation transducers 126 desired directed at a single target region (such as regions 108, 110, 112), such as, but not limited to one, two, three, four, or more. It is further contemplated that the ablation transducers 126 may not be present as an even number or in pairs. The transducers 126 may be arranged in any manner desired.

In some embodiments, the ablation transducers 126 may be positioned at an angle or tilted to more efficiently radiate acoustic energy at a desired location. In some instances, the first pair of transducers 128a, 128b may be positioned at a first angle relative to a longitudinal axis of the elongate shaft 114 such that the acoustic energy 134 radiated from the transducers 128a, 128b is directed towards a shallow region 108 of the desired treatments region 106. The second pair of transducers 130a, 130b may be positioned at a second angle relative to a longitudinal axis of the elongate shaft 114 such that the acoustic energy 136 radiated from the transducers 130a, 130b is directed towards a middle region 112 of the desired treatments region 106. The third pair of transducers 132a, 132b may be positioned at a third angle relative to a longitudinal axis of the elongate shaft 114 such that the acoustic energy 138 radiated from the transducers 132a, 132b is directed towards a deeper region 110 of the desired treatments region 106. In some instances, the first, second, and third angles may be different from one another, while in other instances, the first, second, and third angles may be the same, while in yet other instances, some angles may be the same while others are different. The angles at which the transducers 126 are positioned may vary depending on the size and shape of the desired treatment region. It is contemplated that in some instances, the ablation transducers 126 may be all focused at a single location. In this instance, it may be desirable to direct the ultrasound energy towards the deepest location 110 of the target zone 106. As the tissue is ablated, the tissue attenuation may gradually work the ablation zone back towards the shallowest depth of the focal zone.

While not explicitly shown, the ablation transducers 126 may be connected to a control unit (such as control unit 18 in FIG. 1) by electrical conductor(s). In some embodiments, the electrical conductor(s) may be disposed within a lumen of the elongate shaft 114. In other embodiments, the electrical conductor(s) may extend along an outside surface of the elongate shaft 114. The electrical conductor(s) may provide electricity to the transducers 126 which may then be converted into acoustic energy. The acoustic energy may be directed from the transducers 126 in a direction generally perpendicular to the radiating surfaces of the transducers 126, as illustrated at dashed lines 134, 136, 138. As discussed above, acoustic energy radiates from the transducers 126 in a pattern related to the shape of the transducers 126 and lesions formed during ablation take shape similar to contours of the pressure distribution.

The modulation system 100 may be configured to ablate deeper target tissue 110 first to avoid attenuation problems associated with targeting a shallower region 108 first. In some embodiments each ablation transducer 128a, 128b, 130a, 130b, 132a, 132b may be individually connected to a control unit with separate electrical conductors. In other instances, each pair of transducers 128a,b, 130a,b, 132a,b may be connected to the control unit as pairs. For example the first pair of ablation transducers 128a,b may be connected by a first electrical conductor, the second pair of ablation transducers 130a,b may be connected by a second electrical conductor, and the third pair of ablation transducers 132a,b may be connected by a third electrical conductor. It is contemplated that the imaging transducer(s) 124 may be connected to the control unit by one or more separate electrical conductors.

Once the modulation system 100 has been advanced to the treatment region, energy may be supplied to the pairs of ablation transducers 128a,b, 130a,b, 132a,b. In some instances, the pairs of ablation transducers 128a,b, 130a,b, 132a,b may be sequentially activated such that the deepest tissue region 110 is ablated first, followed by the middle region 112, and finally the shallowest region 108. For example, the third pair 132a,b may be activated, followed by the second pair 130a,b, and finally the first pair 128a,b. It is further contemplated that in some instances ablation transducers focused at different depths may be activated simultaneously to ablate a larger volume of the target tissue 106 at once. The optional imaging transducer 124 may detect tissue changes during ablation. In some instances, the imaging transducer 124 may be operated simultaneously with the ablation transducers 126 to provide real-time feedback of the ablation progress. In other embodiments, the imaging transducer 124 may be operated in an alternating fashion (e.g. an ablation/imaging duty cycle) with the ablation transducers 126 such that the imaging transducer 124 and the ablation transducers 126 are not simultaneously active. The amount of energy delivered to the ablation transducers may be determined by the desired treatment as well as the feedback obtained from the imaging transducer 124. It is contemplated that deeper target regions, such as region 110, may require greater power and/or duration than a shallower region, such as region 108.

The modulation system 100 may be advanced through the vasculature in any manner known in the art. For example, system 100 may include a guidewire lumen to allow the system 100 to be advanced over a previously located guidewire. In some embodiments, the modulation system 100 may be advanced, or partially advanced, within a guide sheath such as the sheath 16 shown in FIG. 1. Once the ablation transducers 126 of the modulation system 100 have been placed adjacent to the desired treatment area, positioning mechanisms may be deployed, such as centering baskets 118, 120, if so provided. While not explicitly shown, the ablation transducers 126 and the imaging transducer 124 may be connected to a single control unit or to separate control units (such as control unit 18 in FIG. 1) by electrical conductors. Once the modulation system 100 has been advanced to the treatment region, energy may be supplied to the ablation transducers 126 and the imaging transducer 124. As discussed above, the energy may be supplied to both the ablation transducers 126 and the imaging transducer 124 simultaneously or in an alternating fashion at desired. The amount of energy delivered to the ablation transducers 126 may be determined by the desired treatment as well as the feedback provided by the imaging transducer 124.

In some instances, the elongate shaft 114 may be rotated and additional ablation can be performed at multiple locations around the circumference of the vessel 102. In some instances, a slow automated “rotisserie” rotation can be used to work around the circumference of the vessel 102, or a faster spinning can be used to simultaneously ablate around the entire circumference. The spinning can be accomplished with a distal micro-motor or by spinning a drive shaft from the proximal end. In some embodiments, ultrasound sensor information can be used to selectively turn on and off the ablation transducers to warm any cool spots or accommodate for veins, or other tissue variations. The number of times the elongate shaft 114 is rotated at a given longitudinal location may be determined by the number and size of the ablation transducers 126 on the elongate shaft 114. Once a particular location has been ablated, it may be desirable to perform further ablation procedures at different longitudinal locations. Once the elongate shaft 114 has been longitudinally repositioned, energy may once again be delivered to the ablation transducers 126 and the imaging transducer 124. If necessary, the elongate shaft 114 may be rotated to perform ablation around the circumference of the vessel 102 at each longitudinal location. This process may be repeated at any number of longitudinal locations desired. It is contemplated that in some embodiments, the system 100 may include transducer arrays 122 at various positions along the length of the modulation system 100 such that a larger region may be treated without longitudinal displacement of the elongate shaft 114.

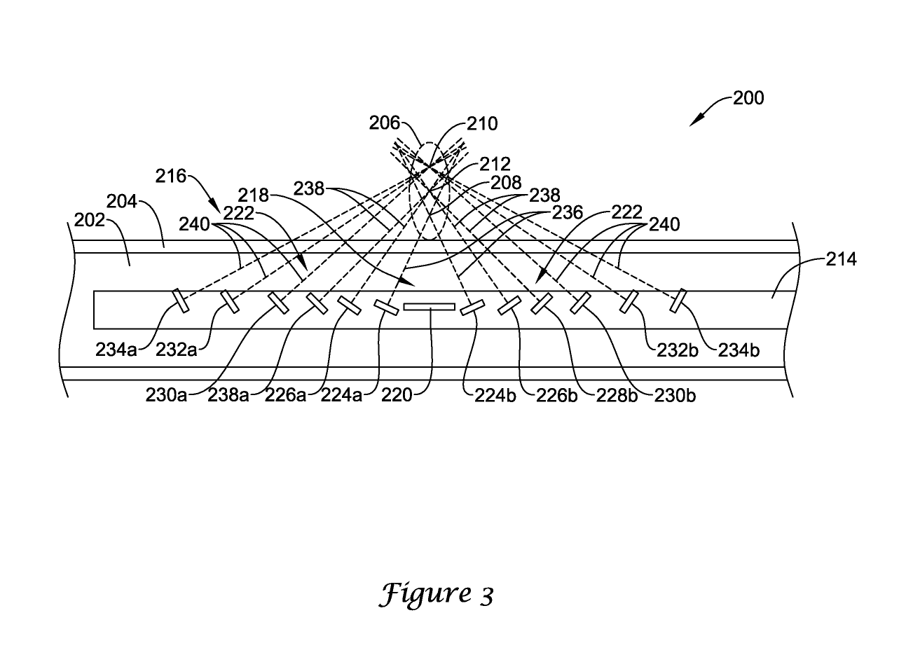

FIG. 3 is an illustrative embodiment of a distal end of a renal nerve modulation system 200 that may be similar in form and function to other systems disclosed herein. The modulation system 200 may be disposed within a body lumen 202 having a vessel wall 204. The vessel wall 204 may be surrounded by local body tissue. The local body tissue may comprise adventitia and connective tissues, nerves, fat, fluid, etc. in addition to the muscular vessel wall 204. A portion of the tissue may be the desired treatment region 206 having a shallow region 208 adjacent to the vessel wall 204, a deeper region 210, and a middle region 212 disposed between the shallow region 208 and the deeper region 210. As will become more apparent below, it is contemplated that there may be any number of sub-regions within the target region 206. The number of sub-regions may be determined by the number and relative position of ablation transducers disposed on the elongate shaft 214.

The system 200 may include an elongate shaft 214 having a distal end region 216. The elongate shaft 214 may extend proximally from the distal end region 216 to a proximal end configured to remain outside of a patient's body. The proximal end of the elongate shaft 214 may include a hub attached thereto for connecting other treatment devices or providing a port for facilitating other treatments. It is contemplated that the stiffness of the elongate shaft 214 may be modified to form a modulation system 200 for use in various vessel diameters and various locations within the vascular tree. The elongate shaft 214 may further include one or more lumens extending therethrough. For example, the elongate shaft 214 may include a guidewire lumen and/or one or more auxiliary lumens. The lumens may be configured in any way known in the art. While not explicitly shown, the modulation system 200 may further include temperature sensors/wire, an infusion lumen, radiopaque marker bands, fixed guidewire tip, a guidewire lumen, external sheath and/or other components to facilitate the use and advancement of the system 200 within the vasculature.

The system 200 may include an array of transducers 218. In some embodiments, the array may include one or more optional imaging transducers 220 and one or more ultrasound ablation transducers 222 disposed adjacent the distal end region 216. However, the transducer array 218 may be placed at any longitudinal location along the elongate shaft 214 desired. In some embodiments, should one be so provided the one or more imaging transducers 220 may be provided at the center of the array 218 to detect tissue changes during the ablation procedure. However, the imaging transducer 220 may be provided at any location within the array desired. In some instances, the ablation transducers 222 may be placed symmetrically about the imaging transducer 220 such that there is equal number of transducers 222 located proximal to the imaging transducer 220 and distal to the imaging transducer 220. However the ablation transducers 222 may be arranged in any pattern desired. For example, in some instances, there may not be an equal number of ablation transducers 222 disposed on either side of the imaging transducer 220. In some embodiments, the imaging transducer 220 may not be present. It is further contemplated that modulation system may include any number of imaging transducers 220 or ablation transducers 222 desired. In some instances, there may be more than one row of transducers 218 disposed on the elongate shaft 214.

The transducer array 218 may include multiple ultrasound ablation transducers 222 physically directed towards multiple focal points. In some embodiments, more than one ablation transducer 222 may be directed towards a single focal point. In some instances, this arrangement may be more efficient than focusing by phased arrays of linearly arranged transducer elements that are not physically directed at the focal point. It is contemplated that an increased efficiency resulting from multiple ablation transducers physically directed towards single focal point may enable the use of fewer transducers and/or lower power. Deeper tissue may require greater power and/or duration for proper ablation than shallower tissue. If the ablation transducers are power-limited (such as needing more elaborate cooling in order to increase power output), then a greater number of transducers can be focused on a single focal point for deeper ablation than for shallower ablation. In some embodiments, the modulation system 200 may include one pair (a first set) of ultrasound ablation transducers 224a, 224b (collectively 224a,b) directed towards the shallow region 208 of the desired treatment region 206, two pairs (a second set) of ablation transducers 226a, 226b, 228a, 228b (collectively 226a,b, 228a,b) directed towards the middle region 212 of the desired treatment region 206, and three pairs (a third set) of ablation transducers 230a, 230b, 232a, 232b, 234a, 234b (collectively 230a,b, 232a,b, 234a,b) directed towards the deeper region 210 of the desired treatment region 206. While the system 200 is described as having a distinct number of transducer pairs directed towards each treatment region, it is contemplated that each treatment region 208, 210, 212 may have any number of transducers (or pairs of transducers) directed at it, such as, but not limited to: one, two, three, four, or more depending on the number of desired target regions. It is further contemplated that the system 200 may have any number of ablation transducers 222 desired directed at a single target region (such as regions 208, 210, 212), such as, but not limited to one, two, three, four, or more. It is further contemplated that the ablation transducers 222 may not be present as an even number or in pairs. The transducers 222 may be arranged in any manner desired.

In some embodiments, the ablation transducers 222 may be positioned at an angle or tilted to more efficiently radiate acoustic energy at a desired location. For example, each pair of transducers 224a,b, 226a,b, 228a,b, 230a,b, 232a,b, 234a,b may be positioned at an angle relative to a longitudinal axis of the elongate shaft 214 such that the acoustic energy 236 radiated from the first set of transducers 224a,b is directed towards a shallow region 208 of the desired treatments region 206, acoustic energy 238 radiated from the second set of transducers 226a,b, 228a,b is directed towards a middle region 212 of the desired treatments region 206, and acoustic energy 240 radiated from the third set of transducers 230a,b, 232a,b, 234a,b is directed towards a deeper region 210 of the desired treatments region 206. In some instances, the angles may be different from one another, while in other instances, the angles may be the same, while in yet other instances, some angles may be the same while others are different. The angles at which the transducers 222 are positioned may vary depending on the size and shape of the desired treatment region.

While not explicitly shown, the ablation transducers 222 may be connected to a control unit (such as control unit 18 in FIG. 1) by electrical conductor(s). In some embodiments, the electrical conductor(s) may be disposed within a lumen of the elongate shaft 214. In other embodiments, the electrical conductor(s) may extend along an outside surface of the elongate shaft 214. The electrical conductor(s) may provide electricity to the transducers 222 which may then be converted into acoustic energy. The acoustic energy may be directed from the transducers 222 in a direction generally perpendicular to the radiating surfaces of the transducers 222, as illustrated at dashed lines 236, 238, 240. As discussed above, acoustic energy radiates from the transducers 222 in a pattern related to the shape of the transducers 222 and lesions formed during ablation take shape similar to contours of the pressure distribution.

The modulation system 200 may be configured to ablate deeper target tissue 210 first to avoid attenuation problems associated with targeting a shallower region 208 first. In some embodiments each ablation transducer 224a, 224b, 226a, 226b, 228a, 228b, 230a, 230b, 232a, 232b, 234a, 234b may be individually connected to a control unit with separate electrical conductors. In other instances, each pair of transducers 224a,b, 226a,b, 228a,b, 230a,b, 232a,b, 234a,b may be connected to the control unit as pairs. For example the first pair of ablation transducers 224a,b may be connected by a first electrical conductor, the second pair of ablation transducers 226a,b may be connected by a second electrical conductor, the third pair of ablation transducers 228a,b may be connected by a third electrical conductor, and so on. It is contemplated that the imaging transducer(s) 220 may be connected to the control unit by one or more separate electrical conductors.

Once the modulation system 200 has been advanced to the treatment region, it is contemplated that energy may be supplied to the ablation transducers as individual transducers, pairs of transducers, or sets of transducers (corresponding to a desired treatment region). In some instances, the first 224a,b, second 226a,b, 228a,b, and third 230a,b, 232a,b, 234a,b sets of ablation transducers may be sequentially activated such that the deepest tissue region 210 is ablated first, followed by the middle region 212, and finally the shallowest region 208. For example, the third set 230a,b, 232a,b, 234a,b may be activated, followed by the second set 226a,b, 228a,b, and finally the first set 224a,b. It is further contemplated that in some instances ablation transducers focused at different depths may be activated simultaneously to ablate a larger volume of the target tissue 206 at once. The optional imaging transducer 220 may detect tissue changes during ablation. In some instances, the imaging transducer 220 may be operated simultaneously with the ablation transducers 222 to provide real-time feedback of the ablation progress. In other embodiments, the imaging transducer 220 may be operated in an alternating fashion (e.g. an ablation/imaging duty cycle) with the ablation transducers 222 such that the imaging transducer 220 and the ablation transducers 222 are not simultaneously active. The amount of energy delivered to the ablation transducers may be determined by the desired treatment as well as the feedback obtained from the imaging transducer 220. It is contemplated that deeper target regions, such as region 210, may require greater power and/or duration than a shallower region, such as region 208.

The modulation system 200 may be advanced through the vasculature in any manner known in the art. For example, system 200 may include a guidewire lumen to allow the system 200 to be advanced over a previously located guidewire. In some embodiments, the modulation system 200 may be advanced, or partially advanced, within a guide sheath such as the sheath 16 shown in FIG. 1. Once the ablation transducers 222 of the modulation system 200 have been placed adjacent to the desired treatment area, positioning mechanisms may be deployed, such as centering baskets, if so provided. While not explicitly shown, the ablation transducers 222 and the imaging transducer 220 may be connected to a single control unit or to separate control units (such as control unit 18 in FIG. 1) by electrical conductors. Once the modulation system 200 has been advanced to the treatment region, energy may be supplied to the ablation transducers 222 and the imaging transducer 220. As discussed above, the energy may be supplied to both the ablation transducers 222 and the imaging transducer 220 simultaneously or in an alternating fashion as desired. The amount of energy delivered to the ablation transducers 222 may be determined by the desired treatment as well as the feedback provided by the imaging transducer 220.

In some instances, the elongate shaft 214 may be rotated and additional ablation can be performed at multiple locations around the circumference of the vessel 202. In some instances, a slow automated “rotisserie” rotation can be used to work around the circumference of the vessel 202, or a faster spinning can be used to simultaneously ablate around the entire circumference. The spinning can be accomplished with a distal micro-motor or by spinning a drive shaft from the proximal end. In some embodiments, ultrasound sensor information can be used to selectively turn on and off the ablation transducers to warm any cool spots or accommodate for veins, or other tissue variations. The number of times the elongate shaft 214 is rotated at a given longitudinal location may be determined by the number and size of the ablation transducers 222 on the elongate shaft 214. Once a particular location has been ablated, it may be desirable to perform further ablation procedures at different longitudinal locations. Once the elongate shaft 214 has been longitudinally repositioned, energy may once again be delivered to the ablation transducers 222 and the imaging transducer 220. If necessary, the elongate shaft 214 may be rotated to perform ablation around the circumference of the vessel 202 at each longitudinal location. This process may be repeated at any number of longitudinal locations desired. It is contemplated that in some embodiments, the system 200 may include transducer arrays 218 at various positions along the length of the modulation system 200 such that a larger region may be treated without longitudinal displacement of the elongate shaft 214.

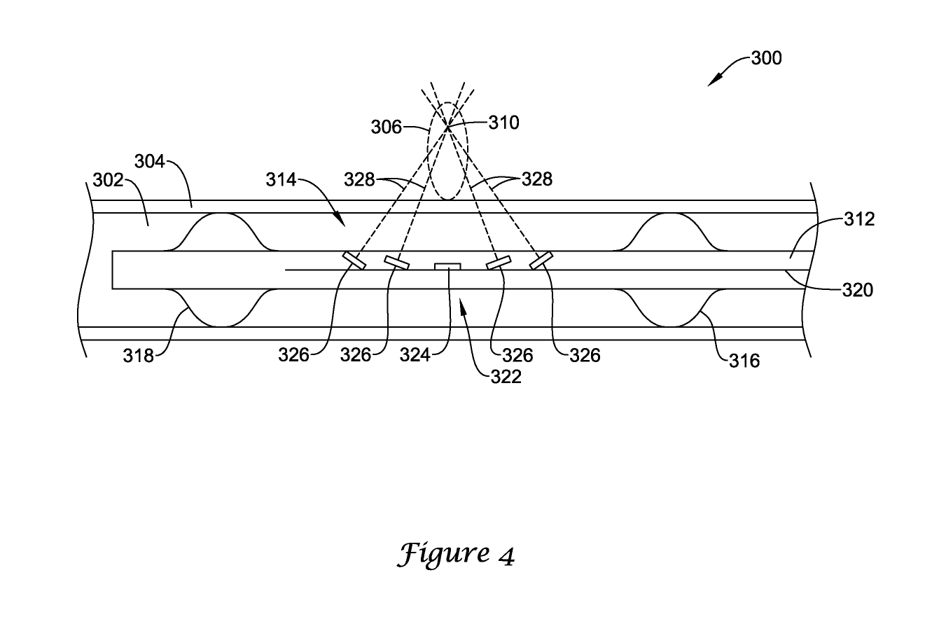

FIG. 4 is an illustrative embodiment of a distal end of a renal nerve modulation system 300 that may be similar in form and function to other systems disclosed herein. The modulation system 300 may be disposed within a body lumen 302 having a vessel wall 304. The vessel wall 304 may be surrounded by local body tissue. The local body tissue may comprise adventitia and connective tissues, nerves, fat, fluid, etc. in addition to the muscular vessel wall 304. A portion of the tissue may be the desired treatment region 306 having a shallow region 308 adjacent to the vessel wall 304 and a deeper region 310. As will become more apparent below, it is contemplated that there may be any number of sub-regions within the target region 306. The number of sub-regions may be determined by the number and relative position of ablation transducers disposed on the elongate shaft 312.

The system 300 may include an elongate shaft 312 having a distal end region 314. The elongate shaft 312 may extend proximally from the distal end region 314 to a proximal end configured to remain outside of a patient's body. The proximal end of the elongate shaft 312 may include a hub attached thereto for connecting other treatment devices or providing a port for facilitating other treatments. It is contemplated that the stiffness of the elongate shaft 312 may be modified to form a modulation system 300 for use in various vessel diameters and various locations within the vascular tree. The elongate shaft 312 may further include one or more lumens extending therethrough. For example, the elongate shaft 312 may include a guidewire lumen and/or one or more auxiliary lumens. The lumens may be configured in any way known in the art. While not explicitly shown, the modulation system 300 may further include temperature sensors/wire, an infusion lumen, radiopaque marker bands, fixed guidewire tip, a guidewire lumen, external sheath and/or other components to facilitate the use and advancement of the system 300 within the vasculature.

The modulation system 300 may include one or more expandable centering baskets or framework 316, 318 disposed adjacent the distal end region 314. In some instances the modulation system 300 may include expandable balloon(s) in place of the expandable basket(s) 316, 318. It is contemplated that a first expandable basket 318 may be positioned distal to the transducer array 322 and a second centering basket 316 may be positioned proximal to the transducer array 322. In some embodiments, the modulation system 300 may further include an actuatable element 320 such as, but not limited to a centering wire or flexing ribbon extending along the elongate member 312. The actuatable element 320 may be configured to extend proximally from the distal end region 314 to a location external to a patient's body. As will be discussed in more detail below, in some embodiments, the array of transducers 322 may be affixed to the actuatable element 320 such that push-pull actuation of actuatable element 320 may adjust the position of the transducers 322 to target a particular location and/or to adjust focus depth. A centering wire may have a thin diameter smaller than a cross-sectional surface area of the transducers 322. The centering wire may be connected to the transducer 322 such that the centering wire is affixed across the center of the cross-section (for example, across the diameter of a circular cross-section) along one of the radiating surfaces of the transducer 322. A flexing ribbon may have a relatively thin width and a depth similar in size to the cross-section of the transducers 322. The centering wire may be connected to the transducer 322 such that the flexing ribbon is disposed over substantially an entire radiating surface of the transducer 322. It is contemplated that structures other than a ribbon or wire may be used to achieve the desired manipulation of the transducers 322.

The system 300 may include an array of transducers 322. In some embodiments, the array may include one or more optional imaging transducers 324 and one or more ultrasound ablation transducers 326 disposed adjacent the distal end region 314. However, the transducer array 322 may be placed at any longitudinal location along the elongate shaft 312 desired. In some embodiments, should one be so provided the one or more imaging transducers 324 may be provided at the center of the array 322 to detect tissue changes during the ablation procedure. However, the imaging transducer 324 may be provided at any location within the array desired. In some instances, the ablation transducers 326 may be placed symmetrically about the imaging transducer 324 such that there is equal number of ablation transducers 326 located proximal to and distal to the imaging transducer 324. However, the ablation transducers 326 may be arranged in any pattern desired. For example, in some instances, there may not be an equal number of ablation transducers 326 disposed on either side of the imaging transducer 324. While the system 300 is illustrated as having four ablation transducers 326, it is contemplated that the modulation system 300 may include any number of ablation transducers 326 desired, such as, but not limited to: one, two, three, five, or more. It is further contemplated that in some embodiments, the imaging transducer 324 may not be present. In some instances, the transducers 322 may be arranged in more than one row on the elongate shaft 314

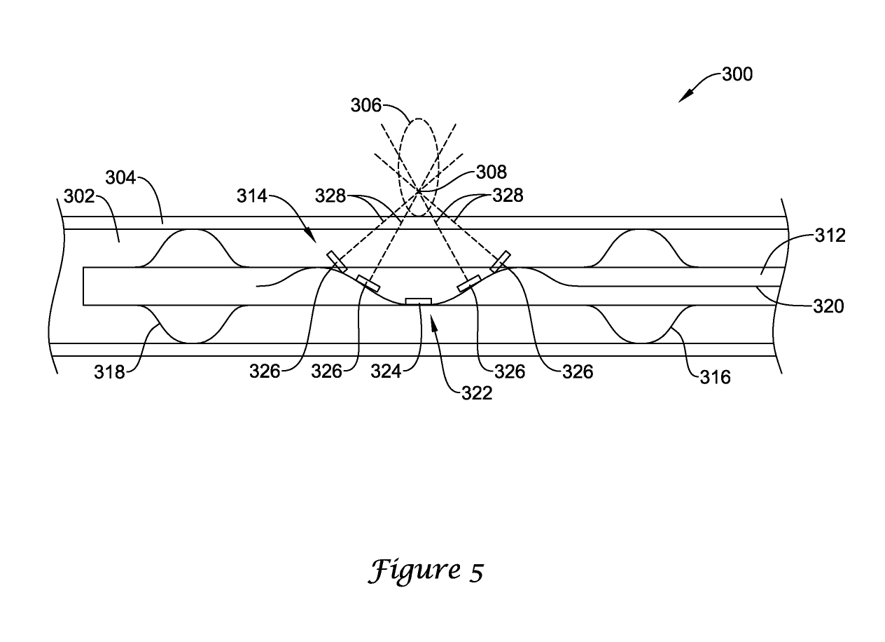

The transducer array 322 may include multiple ultrasound ablation transducers 326 physically directed towards a focal point. In some instances, each ablation transducer 326 may be positioned such that they are all directed towards the same focal point, such as the deeper target region 310. It is contemplated that an increased efficiency resulting from multiple ablation transducers physically directed towards single focal point may enable the use of fewer transducers and/or lower power. As ablated tissue may attenuate ultrasound energy more than unablated tissue, deeper tissue may require greater power and/or duration for proper ablation than shallower tissue as the shallower tissue may be typically ablated first. If the ablation transducers are power-limited (such as needing more elaborate cooling in order to increase power output), then a greater number of transducers can be focused on a single focal point for deeper ablation than for shallower ablation. As discussed above, the transducer array 322 may be affixed to an actuatable element 320. The actuatable element 320 may respond to push-pull actuation causing the actuatable element 320 to change shape, thus changing the orientation and the focal point of the transducer array 322. While not explicitly shown, it is contemplated that the elongate shaft 312 may flex with the actuatable element 320. In some instances, the actuatable element 320 may be disposed within a lumen of the elongate shaft 312. In other instances, the actuatable element 320 may be affixed to an outer surface of the elongate shaft 312. Flexing of the actuatable element 320 may change the angle of the ablation transducers 326 such that they are physically directed towards a different focal point, such as the shallow target region 308 as shown in FIG. 5, than when the actuatable element 320 is in an unflexed state. While the actuatable element 320 is illustrated as curved to two sides, it is contemplated that the actuatable element 320 may be configured to flex on a single side. This may allow the user to target deeper target tissue 310 first, followed by shallower target tissue 308 thus minimizing or eliminating tissue attenuation problems. It is contemplated that the actuatable element 320 may be flexed to focus the ablation transducers 326 through a continuous range of depths. For example, an increasing force may be applied to the actuatable element 320 such that the actuatable element 320 is continuously moving the ablation transducers 326 from targeting a deep location to a location just outside the vessel wall 304. In other instances, the actuatable element 320 may be incrementally flexed such that the ablation transducers 326 are focused on discrete locations.

It is contemplated that the transducer array 322 can be mounted on the actuatable element 320 in a number of ways. In one instance, the ablation transducers 326 may be mounted to the actuatable element 320 at various angles as shown in FIG. 4. In such a configuration, the ablation transducers 326 may be focused at the deepest target region, such as target region 310, and flexing the actuatable element 320 focuses the energy closer to the vessel wall 304. This arrangement may require less overall displacement of the actuatable element 320 to achieve the required range of focus compared to other transducer 326 orientations. In other instances, the transducers 326 may be mounted flat on the actuatable element 320. This arrangement may require more overall displacement of the actuatable element 320 to achieve focusing at all depths. While the actuatable element 320 is referred to as a single element, it is contemplated that the actuatable element 320 may be formed of multiple ribbons or wires either joined or separate. In other embodiments, one or more actuatable elements 320 may be affixed to the side(s) or perimeter of the transducers 322.

While not explicitly shown, the ablation transducers 326 may be connected to a control unit (such as control unit 18 in FIG. 1) by electrical conductor(s). In some embodiments, the electrical conductor(s) may be disposed within a lumen of the elongate shaft 312. In other embodiments, the electrical conductor(s) may extend along an outside surface of the elongate shaft 312. The electrical conductor(s) may provide electricity to the transducers 326 which may then be converted into acoustic energy. The acoustic energy may be directed from the transducers 326 in a direction generally perpendicular to the radiating surfaces of the transducers 326, as illustrated at dashed lines 328. As discussed above, acoustic energy radiates from the transducers 326 in a pattern related to the shape of the transducers 326 and lesions formed during ablation take shape similar to contours of the pressure distribution.

The modulation system 300 may be configured to ablate deeper target tissue 310 first to avoid attenuation problems associated with targeting a shallower region 308 first. In some embodiments each ablation transducer 326 may be individually connected to a control unit with separate electrical conductors. In other instances, any number of ablation transducers 326 may be connected to a single electrical conductor such as, but not limited to, one, two, three, or four, etc. It is contemplated that the imaging transducer(s) 324 may be connected to the control unit by one or more separate electrical conductors.

Once the modulation system 300 has been advanced to the treatment region, the actuatable element 320 may be flexed, if necessary, to focus the ablation transducers 326 at the desired treatment location and energy is then supplied to the ablation transducers 326. It is contemplated that energy may be supplied to the ablation transducers 326 as individual transducers, pairs of transducers, or sets of transducers. In some instances, the ablation transducers 326 may be activated simultaneously, however this is not required. In some embodiments, the actuatable element 320 may be oriented such that the deepest tissue region 310 is ablated first, followed by the shallowest region 308. As ablation of a desired region is completed, the actuatable element 320 may be actuated to change the focus of the ablation transducers 326. It is contemplated that flexing of the actuatable element 320 may be performed continuously or incrementally, as desired. The ablation transducers 326 may be focused on as many treatment regions as desired and energy supplied to each region. The optional imaging transducer 324 may detect tissue changes during ablation. In some instances, the imaging transducer 324 may be operated simultaneously with the ablation transducers 326 to provide real-time feedback of the ablation progress. In other embodiments, the imaging transducer 324 may be operated in an alternating fashion (e.g. an ablation/imaging duty cycle) with the ablation transducers 326 such that the imaging transducer 324 and the ablation transducers 326 are not simultaneously active. The amount of energy delivered to the ablation transducers 326 may be determined by the desired treatment as well as the feedback obtained from the imaging transducer 324. It is contemplated that deeper target regions, such as region 310, may require greater power and/or duration than a shallower region, such as region 308.

The modulation system 300 may be advanced through the vasculature in any manner known in the art. For example, system 300 may include a guidewire lumen to allow the system 300 to be advanced over a previously located guidewire. In some embodiments, the modulation system 300 may be advanced, or partially advanced, within a guide sheath such as the sheath 16 shown in FIG. 1. Once the ablation transducers 326 of the modulation system 300 have been placed adjacent to the desired treatment area, positioning mechanisms may be deployed, such as centering baskets 316, 318, if so provided. While not explicitly shown, the ablation transducers 326 and the imaging transducer 324 may be connected to a single control unit or to separate control units (such as control unit 18 in FIG. 1) by electrical conductors. Once the modulation system 300 has been advanced to the treatment region, the actuatable element 320 may be actuated as necessary to focus the ablation transducers 326 at a desired region. Energy may then be supplied to the ablation transducers 326 and the imaging transducer 324. As discussed above, the energy may be supplied to both the ablation transducers 326 and the imaging transducer 324 simultaneously or in an alternating fashion at desired. The amount of energy delivered to the ablation transducers 326 may be determined by the desired treatment as well as the feedback provided by the imaging transducer 324. As ablation of a desired region is completed, the actuatable element 320 may be actuated to change the focus of the ablation transducers 326. It is contemplated that flexing of the actuatable element 320 may be performed continuously or incrementally, as desired. The ablation transducers 326 may be focused on as many treatment regions as desired and energy supplied to each region.

In some instances, the elongate shaft 312 may be rotated and additional ablation can be performed at multiple locations around the circumference of the vessel 302. In some instances, a slow automated “rotisserie” rotation can be used to work around the circumference of the vessel 302, or a faster spinning can be used to simultaneously ablate around the entire circumference. The spinning can be accomplished with a distal micro-motor or by spinning a drive shaft from the proximal end. In some embodiments, ultrasound sensor information can be used to selectively turn on and off the ablation transducers to warm any cool spots or accommodate for veins, or other tissue variations. The number of times the elongate shaft 312 is rotated at a given longitudinal location may be determined by the number and size of the ablation transducers 326 on the elongate shaft 312. Once a particular location has been ablated, it may be desirable to perform further ablation procedures at different longitudinal locations. Once the elongate shaft 312 has been longitudinally repositioned, energy may once again be delivered to the ablation transducers 326 and the imaging transducer 324. If necessary, the elongate shaft 312 may be rotated to perform ablation around the circumference of the vessel 302 at each longitudinal location. This process may be repeated at any number of longitudinal locations desired. It is contemplated that in some embodiments, the system 300 may include transducer arrays 322 at various positions along the length of the modulation system 300 such that a larger region may be treated without longitudinal displacement of the elongate shaft 312.

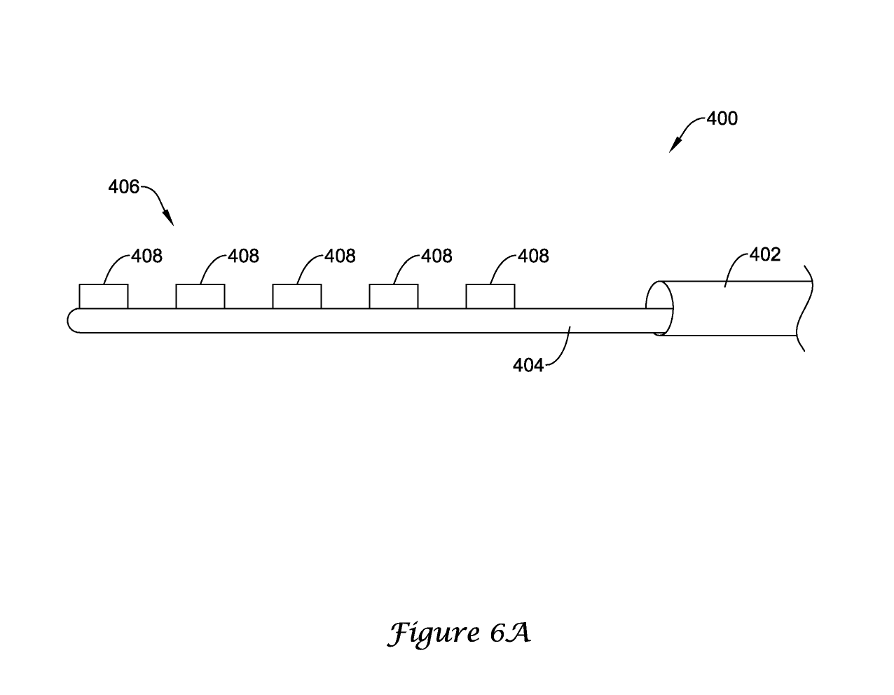

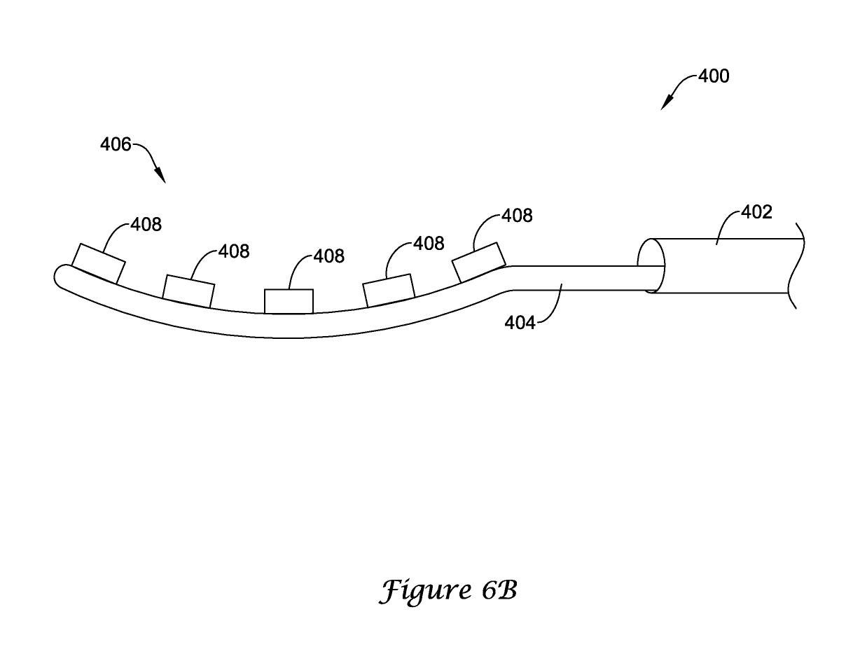

FIGS. 6A and 6B are an illustrative embodiment of a distal end of a renal nerve modulation system 400 that may be similar in form and function to other systems disclosed herein. The system 400 may include a catheter shaft 402 having a lumen. The catheter shaft 402 may function as delivery sheath for an elongate shaft 404 and transducers 408. The elongate shaft 404 may extend proximally within the lumen of the catheter shaft 402 from a distal end region 406 to a proximal end configured to remain outside of a patient's body. In some embodiments, the catheter shaft 402 may not be provided. The proximal end of the elongate shaft 404 and/or catheter shaft 402 may include a hub attached thereto for connecting other treatment devices or providing a port for facilitating other treatments. It is contemplated that the stiffness of the elongate shaft 404 may be modified to form a modulation system 400 for use in various vessel diameters and various locations within the vascular tree. The elongate shaft 404 may further include one or more lumens extending therethrough. For example, the elongate shaft 404 may include a guidewire lumen and/or one or more auxiliary lumens. The lumens may be configured in any way known in the art. While not explicitly shown, the modulation system 400 may further include temperature sensors/wire, an infusion lumen, radiopaque marker bands, fixed guidewire tip, a guidewire lumen, external sheath and/or other components to facilitate the use and advancement of the system 400 within the vasculature.

The system 400 may include an array of transducers 408 disposed adjacent the distal end region 406 of the elongate shaft 404. In some embodiments, the array may include one or more optional imaging transducers (not explicitly shown) and one or more ultrasound ablation transducers 408 disposed adjacent the distal end region 406. However, the transducer array 408 may be placed at any longitudinal location along the elongate shaft 404 desired. In some embodiments, should one be so provided the one or more imaging transducers may be provided at the center of the array 408 to detect tissue changes during the ablation procedure. However, the imaging transducer may be provided at any location within the array desired. In some instances, the ablation transducers 408 may be placed symmetrically about the imaging transducer such that there is equal number of ablation transducers 408 located proximal to and distal to the imaging transducer. However, the ablation transducers 408 may be arranged in any pattern desired. For example, in some instances, there may not be an equal number of ablation transducers 408 disposed on either side of the imaging transducer. It is further contemplated that in some embodiments, an imaging transducer may not be present. While the system 400 is illustrated as having five transducers 408, it is contemplated that the modulation system 400 may include any number of ablation and/or imaging transducers 408 desired, such as, but not limited to: one, two, three, four, or more. It is further contemplated that more than one row of transducers 408 may be disposed on the elongate shaft 404.

The transducer array 408 may include multiple ultrasound ablation transducers 408 configured to be physically directed towards a focal point. In some instances, the distal end region 406 of the elongate shaft 404 may be formed from a shape memory material. Suitable shape memory materials may include metals such as nitinol or shape memory polymers. It is contemplated that in some embodiments the distal end region 406 may be formed from any material capable of moving from at least a first configuration to a second configuration upon application of a stimulus such as heat (in some instances body heat may be sufficient) or electricity. In a first configuration, the elongate member 404 may have a linear or substantially linear configuration, as shown in FIG. 6A. Such a configuration may decrease the delivery profile of the modulation system 400. In a second configuration, the distal end region 406 of the elongate shaft 404 may be curved such that each ablation transducer 408 is directed towards a focal point, as shown in FIG. 6B. In some instances, more than one ablation transducer may be directed towards the same focal point, although this is not required. While the modulation system 400 is described as having two configurations, it is contemplated that the modulation system 400 may have any number of configurations desired to perform the desired ablation.

It is contemplated that an increased efficiency resulting from multiple ablation transducers physically directed towards single focal point may enable the use of fewer transducers and/or lower power. As ablated tissue may attenuate ultrasound energy more than unablated tissue, deeper tissue may require greater power and/or duration for proper ablation than shallower tissue as the shallower tissue may be typically ablated first. If the ablation transducers are power-limited (such as needing more elaborate cooling in order to increase power output), then a greater number of transducers can be focused on a single focal point for deeper ablation than for shallower ablation.

While not explicitly shown, the ablation transducers 408 may be connected to a control unit (such as control unit 18 in FIG. 1) by electrical conductor(s). In some embodiments, the electrical conductor(s) may be disposed within a lumen of the elongate shaft 404. In other embodiments, the electrical conductor(s) may extend along an outside surface of the elongate shaft 404. The electrical conductor(s) may provide electricity to the transducers 408 which may then be converted into acoustic energy. The acoustic energy may be directed from the transducers 408 in a direction generally perpendicular to the radiating surfaces of the transducers 408. As discussed above, acoustic energy radiates from the transducers 408 in a pattern related to the shape of the transducers 408 and lesions formed during ablation take shape similar to contours of the pressure distribution.

The modulation system 400 may be configured to ablate deeper target tissue first to avoid attenuation problems associated with targeting a shallower region first. In some embodiments each ablation transducer 408 may be individually connected to a control unit with separate electrical conductors. In other instances, any number of ablation transducers 408 may be connected to a single electrical conductor such as, but not limited to, one, two, three, or four, etc. It is contemplated that the imaging transducer(s), should one be so provided, may be connected to the control unit by one or more separate electrical conductors.

Once the modulation system 400 has been advanced to the treatment region, energy is then supplied to the ablation transducers 408. It is contemplated that energy may be supplied to the ablation transducers 408 as individual transducers, pairs of transducers, or sets of transducers. In some embodiments, the ablation transducers may be activated in such a manner that the transducers directed towards deeper tissue are activated first. It is contemplated that the ablation transducers 408 may be sequentially activated such that ablation is performed from the deepest target tissue to the shallowest target tissue. However, this is not required. It is further contemplated that in some instances ablation transducers focused at different depths may be activated simultaneously to ablate a larger volume of the target tissue at once. The optional imaging transducer may detect tissue changes during ablation. In some instances, the imaging transducer may be operated simultaneously with the ablation transducers 408 to provide real-time feedback of the ablation progress. In other embodiments, the imaging transducer may be operated in an alternating fashion (e.g. an ablation/imaging duty cycle) with the ablation transducers 408 such that the imaging transducer and the ablation transducers 408 are not simultaneously active. The amount of energy delivered to the ablation transducers 408 may be determined by the desired treatment as well as the feedback obtained from the imaging transducer. It is contemplated that deeper target regions may require greater power and/or duration than a shallower region.

The modulation system 400 may be advanced through the vasculature in any manner known in the art. For example, system 400 may include a guidewire lumen to allow the system 400 to be advanced over a previously located guidewire. In some embodiments, the modulation system 400 may be advanced, or partially advanced, within a guide sheath or catheter 402. Once the ablation transducers 408 of the modulation system 400 have been placed adjacent to the desired treatment area, positioning mechanisms may be deployed, such as centering baskets, if so provided. While not explicitly shown, the ablation transducers 408 may be connected to a single control unit or to separate control units (such as control unit 18 in FIG. 1) by electrical conductors. Once the modulation system 400 has been advanced to the treatment region, the distal end region 406 may be actuated as necessary to focus the ablation transducers 408 at a desired region. Energy may then be supplied to the ablation transducers 408. As discussed above, the energy may be supplied to both the ablation transducers 408 and the imaging transducer simultaneously or in an alternating fashion as desired. The amount of energy delivered to the ablation transducers 408 may be determined by the desired treatment as well as the feedback provided by the imaging transducer. As ablation of a desired region is completed, the distal end region 406 may be actuated to change the focus of the ablation transducers 408 and/or different ablation transducers may be activated to target a different region.

In some instances, the elongate shaft 404 may be rotated and additional ablation can be performed at multiple locations around the circumference of the vessel. In some instances, a slow automated “rotisserie” rotation can be used to work around the circumference of the vessel, or a faster spinning can be used to simultaneously ablate around the entire circumference. The spinning can be accomplished with a distal micro-motor or by spinning a drive shaft from the proximal end. In some embodiments. ultrasound sensor information can be used to selectively turn on and off the ablation transducers to warm any cool spots or accommodate for veins, or other tissue variations. The number of times the elongate shaft 404 is rotated at a given longitudinal location may be determined by the number and size of the ablation transducers 408 on the elongate shaft 404. Once a particular location has been ablated, it may be desirable to perform further ablation procedures at different longitudinal locations. Once the elongate shaft 404 has been longitudinally repositioned, energy may once again be delivered to the ablation transducers 408 and the imaging transducer. If necessary, the elongate shaft 404 may be rotated to perform ablation around the circumference of the vessel at each longitudinal location. This process may be repeated at any number of longitudinal locations desired. It is contemplated that in some embodiments, the system 400 may include transducer arrays 408 at various positions along the length of the modulation system 400 such that a larger region may be treated without longitudinal displacement of the elongate shaft 404.

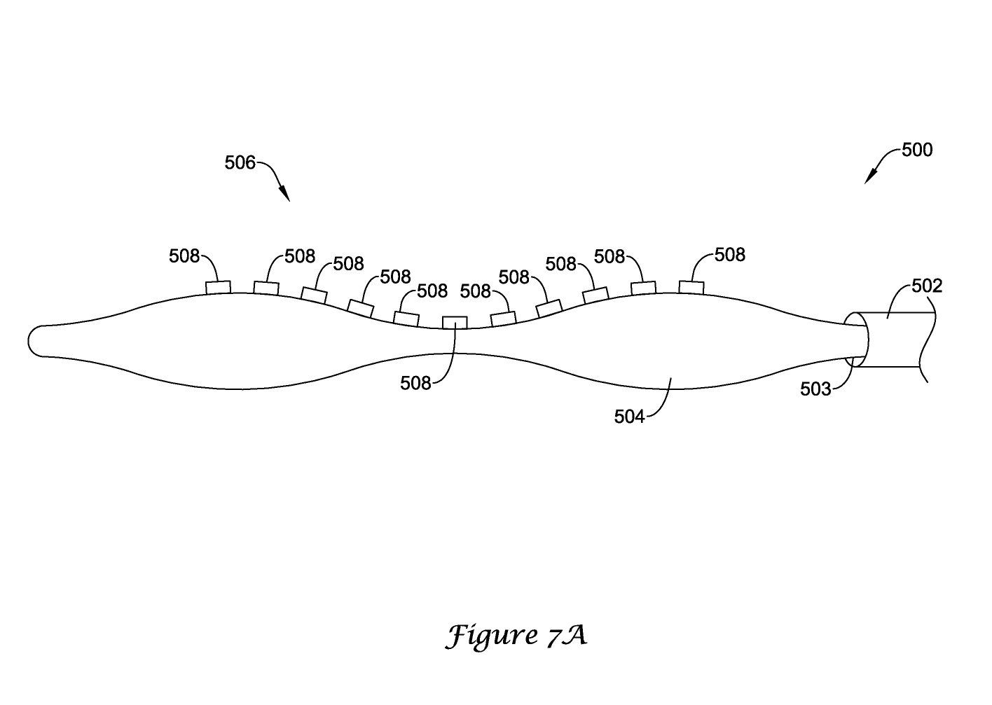

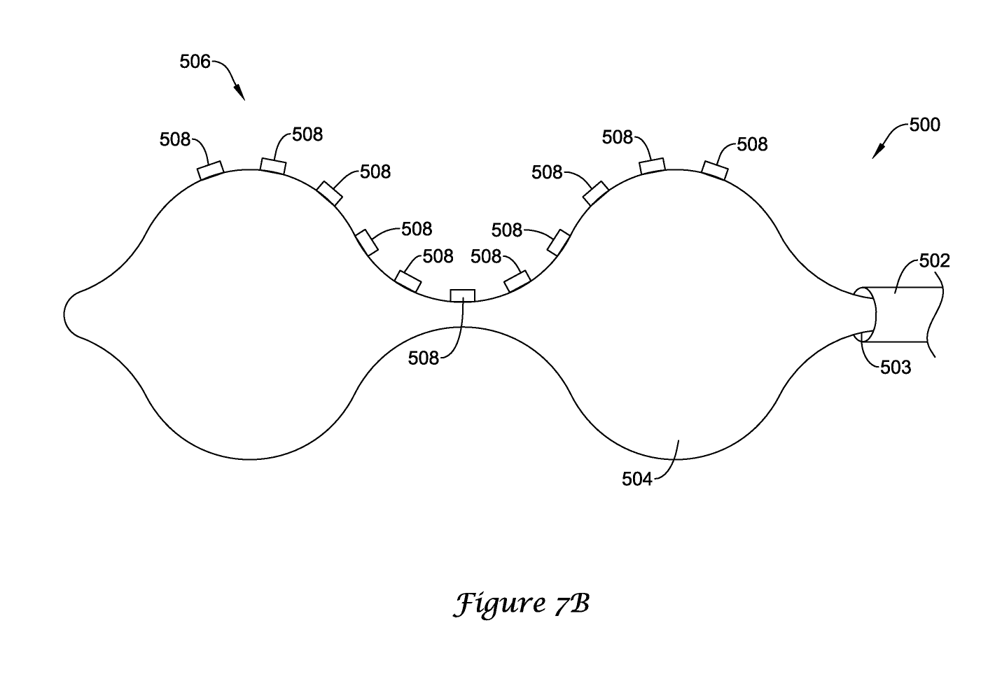

FIGS. 7A and 7B are an illustrative embodiment of a distal end of a renal nerve modulation system 500 that may be similar in form and function to other systems disclosed herein. The system 500 may include a catheter shaft 502 having a lumen. The catheter shaft 502 may function as delivery sheath for an elongate shaft 503. Alternatively, or additionally, the lumen of the catheter shaft 502 may be used to perfuse a fluid, such as, but not limited to a cooling fluid, into a vessel lumen. An elongate shaft 503 may extend proximally within the lumen of the catheter shaft 502 from a distal end region 506 to a proximal end configured to remain outside of a patient's body. The catheter shaft 503 may further include an inflatable member or balloon 504 disposed adjacent the distal end region 506. In some embodiments, the catheter shaft 502 may not be provided. The proximal end of the elongate shaft and/or catheter shaft 502 may include a hub attached thereto for connecting other treatment devices or providing a port for facilitating other treatments. It is contemplated that the stiffness of the elongate shaft 503 may be modified to form a modulation system 500 for use in various vessel diameters and various locations within the vascular tree. The elongate shaft 503 may further include one or more lumens extending therethrough. For example, the elongate shaft 503 may include a guidewire lumen and/or one or more auxiliary lumens. The lumens may be configured in any way known in the art. While not explicitly shown, the modulation system 500 may further include temperature sensors/wire, an infusion lumen, radiopaque marker bands, fixed guidewire tip, a guidewire lumen, external sheath and/or other components to facilitate the use and advancement of the system 500 within the vasculature.

The system 500 may include an array of transducers 508 disposed on the inflatable balloon 504. In some embodiments, the array may include one or more optional imaging transducers (not explicitly shown) and one or more ultrasound ablation transducers 508 disposed on an outer surface of an inflatable balloon 504. However, the transducer array 508 and/or inflatable balloon 504 may be placed at any longitudinal location along the elongate shaft 503 desired. It is further contemplated, that in some instances, the one or more ablation transducers 508 may be placed inside the balloon 504. Such a configuration may allow for cooling of the vessel wall, centering of the transducers, cooling of the transducers, and/or other benefits. In some embodiments, should one be so provided the one or more imaging transducers may be provided at the center of the array 508 to detect tissue changes during the ablation procedure. However, the imaging transducer may be provided at any location within the array desired. In some instances, the ablation transducers 508 may be placed symmetrically about the imaging transducer such that there is equal number of ablation transducers 508 located proximal to and distal to the imaging transducer. However, the ablation transducers 508 may be arranged in any pattern desired. For example, in some instances, there may not be an equal number of ablation transducers 508 disposed on either side of the imaging transducer. It is further contemplated that in some embodiments, an imaging transducer may not be present. While the system 500 is illustrated as having eleven transducers 508, it is contemplated that the modulation system 500 may include any number of ablation and/or imaging transducers 508 desired, such as, but not limited to: one, two, three, four, or more. It is further contemplated that more than one row of transducers 508 may be disposed on the balloon 504.

The transducer array 508 may include multiple ultrasound ablation transducers 508 configured to be physically directed towards a focal point. In some instances, the inflatable balloon 504 may be shaped such that when it is inflated, the ablation transducers 508 are directed towards one or more focal points. In some instances, more than one ablation transducer may be directed towards the same focal point, although this is not required. It is contemplated that the focal position of the transducers 508 may be manipulated by changing the volume of inflation fluid within the inflatable balloon 504. For example, FIG. 7A illustrates a partially inflated balloon 504. As acoustic energy is radiated perpendicular to the surface of the transducer 508, the focal point of the transducers 508 can be changed by changing the angle of the transducer. Further inflation of the balloon 504 may change the angle of the transducers 508 thus changing the focal point, as illustrated in FIG. 7B. For illustrative purposes, the degree to which the inflatable balloon 504 has been inflated in FIG. 7B may be exaggerated from typical use. In some embodiments, the inflatable balloon 504 may have an hourglass shape. Such a shape may provide symmetry to the transducer array 508 and may allow pairs of transducers to target the same focal point, as illustrated in FIGS. 2, 3, 4 and 5. However, it is contemplated that the balloon 504 may take any shape desired. In some embodiments, the balloon 504 may be sized such that, even when fully inflated, the balloon 504 does not occlude the lumen. This may allow blood to continue to flow through the lumen during the ablation procedure. However, in some instances, the inflated balloon 504 may occlude the lumen.

It is contemplated that an increased efficiency resulting from multiple ablation transducers physically directed towards single focal point may enable the use of fewer transducers and/or lower power. As ablated tissue may attenuate ultrasound energy more than unablated tissue, deeper tissue may require greater power and/or duration for proper ablation than shallower tissue as the shallower tissue may be typically ablated first. If the to ablation transducers are power-limited (such as needing more elaborate cooling in order to increase power output), then a greater number of transducers can be focused on a single focal point for deeper ablation than for shallower ablation.

While not explicitly shown, the ablation transducers 508 may be connected to a control unit (such as control unit 18 in FIG. 1) by electrical conductor(s). In some embodiments, the electrical conductor(s) may be disposed within a lumen of the elongate shaft 503. In other embodiments, the electrical conductor(s) may extend along an outside surface of the elongate shaft 503. The electrical conductor(s) may provide electricity to the transducers 508 which may then be converted into acoustic energy. The acoustic energy may be directed from the transducers 508 in a direction generally perpendicular to the radiating surfaces of the transducers 508. As discussed above, acoustic energy radiates from the transducers 508 in a pattern related to the shape of the transducers 508 and lesions formed during ablation take shape similar to contours of the pressure distribution.

The modulation system 500 may be configured to ablate deeper target tissue first to avoid attenuation problems associated with targeting a shallower region first. In some embodiments each ablation transducer 508 may be individually connected to a control unit with separate electrical conductors. In other instances, any number of ablation transducers 508 may be connected to a single electrical conductor such as, but not limited to, one, two, three, or four, etc. It is contemplated that the imaging transducer(s), should one be so provided, may be connected to the control unit by one or more separate electrical conductors.

Once the modulation system 500 has been advanced to the treatment region, energy is then supplied to the ablation transducers 508. It is contemplated that energy may be supplied to the ablation transducers 508 as individual transducers, pairs of transducers, or sets of transducers. In some embodiments, the ablation transducers may be activated in such a manner that the transducers directed towards deeper tissue are activated first. It is contemplated that the ablation transducers 508 may be sequentially activated such that ablation is performed from the deepest target tissue to the shallowest target tissue. However, this is not required. It is further contemplated that in some instances ablation transducers focused at different depths may be activated simultaneously to ablate a larger volume of the target tissue at once. The optional imaging transducer may detect tissue changes during ablation. In some instances, the imaging transducer may be operated simultaneously with the ablation transducers 508 to provide real-time feedback of the ablation progress. In other embodiments, the imaging transducer may be operated in an alternating fashion (e.g. an ablation/imaging duty cycle) with the ablation transducers 508 such that the imaging transducer and the ablation transducers 508 are not simultaneously active. The amount of energy delivered to the ablation transducers 508 may be determined by the desired treatment as well as the feedback obtained from the imaging transducer. It is contemplated that deeper target regions may require greater power and/or duration than a shallower region.

The modulation system 500 may be advanced through the vasculature in any manner known in the art. For example, system 500 may include a guidewire lumen to allow the system 500 to be advanced over a previously located guidewire. In some embodiments, the modulation system 500 may be advanced, or partially advanced, within a guide sheath or catheter 502. Once the ablation transducers 508 of the modulation system 500 have been placed adjacent to the desired treatment area, the balloon member 504 may be inflated. Energy may then be supplied to the ablation transducers 508. While not explicitly shown, the ablation transducers 508 may be connected to a single control unit or to separate control units (such as control unit 18 in FIG. 1) by electrical conductors. As discussed above, the energy may be supplied to both the ablation transducers 508 and the imaging transducer simultaneously or in an alternating fashion at desired. The amount of energy delivered to the ablation transducers 508 may be determined by the desired treatment as well as the feedback provided by the imaging transducer. As ablation of a desired region is completed, the balloon 504 may be inflated to different degrees at the same position as desired to achieve the desired ablation, if necessary.

In some instances, the elongate shaft 503 may be rotated and additional ablation can be performed at multiple locations around the circumference of the vessel. In some instances, a slow automated “rotisserie” rotation can be used to work around the circumference of the vessel, or a faster spinning can be used to simultaneously ablate around the entire circumference. The spinning can be accomplished with a distal micro-motor or by spinning a drive shaft from the proximal end. In some embodiments, ultrasound sensor information can be used to selectively turn on and off the ablation transducers to warm any cool spots or accommodate for veins, or other tissue variations. The number of times the elongate shaft 503 is rotated at a given longitudinal location may be determined by the number and size of the ablation transducers 508 on the elongate shaft 503. Once a particular location has been ablated, it may be desirable to perform further ablation procedures at different longitudinal locations. Once the elongate shaft 503 has been longitudinally repositioned, energy may once again be delivered to the ablation transducers 508 and the imaging transducer. If necessary, the elongate shaft 503 may be rotated to perform ablation around the circumference of the vessel at each longitudinal location. This process may be repeated at any number of longitudinal locations desired. It is contemplated that in some embodiments, the system 500 may include transducer arrays 508 at various positions along the length of the modulation system 500 such that a larger region may be treated without longitudinal displacement of the elongate shaft 503.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. An intravascular nerve modulation system, comprising:
an elongate shaft having a proximal end region and a distal end region; and
an array of ultrasound ablation transducers disposed at the distal end region;
wherein each of the ablation transducers in the array are configured to emit acoustic energy directed towards and intersecting at a first focal region.

2. The system of claim 1, further comprising at least one imaging transducer.

3. The system of claim 1, further comprising a second array of ultrasound ablation transducers directed towards a second focal region.

4. The system of claim 1, wherein the array of ultrasound ablation transducers comprises at least a first set of ablation transducers and a second set of ablation transducers.

5. The system of claim 4, wherein the first set of ablation transducers are positioned at a first angle relative to a longitudinal axis of the elongate shaft and the second set of ablation transducers are positioned at a second angle relative to the longitudinal axis of the elongate shaft.

6. The system of claim 5, wherein the first angle is different than the second angle.

7. The system of claim 4, wherein the first set of ablation transducers are directed towards a first focal point and the second set of ablation transducers are directed towards a second focal point.

8. The system of claim 1, further comprising a flexing element extending from the distal end region to the proximal end region of the elongate shaft.

9. The system of claim 8, wherein the ablation transducers are affixed to a distal portion of the flexing element.

10. The system of claim 9, wherein flexing of the flexing element changes a focal point of each of the ablation transducers.

11. The system of claim 1, wherein the distal end region of the elongate shaft comprises a shape memory material having a first configuration and a second configuration.

12. The system of claim 11, wherein moving from the first configuration to the second configuration changes a focal point of each of the ablation transducer.

13. The system of claim 1, further comprising an inflatable balloon disposed adjacent the distal end region of the elongate shaft.

14. The system of claim 13, wherein inflating the balloon changes a focal point of each of the ablation transducer.

15. The system of claim 1, further comprising a control unit electrically connected to the ablation transducers.

16. The system of claim 15, wherein electricity is supplied to the ablation transducers such that a deeper target region is ablated before a shallower target region.

17. A nerve modulation system, comprising:
a control unit;
an elongate shaft having a proximal end region and a distal end region;
a first set of ablation transducers electrically connected to the control unit, the first set of ablation transducers disposed at the distal end region of the elongate shaft;
a second set of ablation transducers electrically connected to the control unit, the second set of ablation transducers disposed adjacent to the first set of ablation transducers;
one or more imaging transducers electrically connected to the control unit, the one or more imaging transducers disposed adjacent to the first set of ablation transducers;
wherein the first set of ablation transducers are directed towards a first focal point and the second set of ablation transducers are directed towards a second focal point different from the first focal point.

18. The system of claim 17, wherein electricity is supplied to the first and second sets of ablation transducers such that a deeper target region is ablated before a shallower target region.

19. A nerve modulation system, comprising:
a control unit;
an elongate shaft having a proximal end region and a distal end region;
an actuatable element extending along the elongate shaft from the distal end region to the proximal end region;
a plurality of ablation transducers electrically connected to the control unit, the plurality of ablation transducers affixed to a distal portion of the actuatable element;
one or more imaging transducers electrically connected to the control unit, the one or more imaging transducers disposed adjacent the plurality of ablation transducers;
wherein the actuatable element is actuatable between a first configuration and a second configuration to change a focal point of the plurality of ablation transducers.

20. The system of claim 19, wherein in the first configuration of the actuatable element the plurality of ablation transducers are directed to a first deeper focal point and in the second configuration of the actuatable element the plurality of ablation transducers are directed to a second shallower focal point.
